(19) United States
(12) Patent Application Publication
(10) Pub. No.: US 2023/0128966 A1
(43) Pub. Date: Apr. 27, 2023

(54) SYSTEM, METHOD, AND COMPUTER READABLE STORAGE MEDIUM FOR ACCURATE AND RAPID EARLY DIAGNOSIS OF COVID-19 FROM CHEST X RAY

(71) Applicant: Imam Abdulrahman Bin Faisal University, Dammam (SA)

(72) Inventors: Mahbubunnabi TAMAL, Dammam (SA); Maha ALSHAMMARI, Dammam (SA); Meernah ALABDULLAH, Dammam (SA); Rana HOURANI, Dammam (SA); Hossain Abu ALOLA, Dammam (SA); Tarek M. HEGAZI, Dammam (SA)

(73) Assignee: Imam Abdulrahman Bin Faisal University, Dammam (SA)

(21) Appl. No.: 17/507,309

(22) Filed: Oct. 21, 2021

(51) Int. Cl. G06T 7/10; G16H 50/70; G16H 50/80; G16H 50/20; G16H 30/00

(52) U.S. Cl. CPC G06T 7/10; G16H 50/70; G16H 50/80; G16H 50/20; G16H 30/00; G06T 2207/30061; G06T 2207/10116; G06T 2207/20081; G06T 2207/20084

(57) ABSTRACT

A mobile device, computer readable storage medium and method diagnose COVID-19 from at least one Chest X-Ray image. The method can include imaging, by a chest x-ray machine, a person's chest area to obtain the at least one chest x-ray image, performing image segmentation of a human lung in the at least one Chest X-Ray image; extracting radiomics features from the segmented lung, selecting a subset of the radiomics features for classification ability between two classes of COVID-19 and non-COVID-19 including other lung diseases, classifying between COVID-19 and non-COVID-19 using an ensemble bagged model having a plurality of classifiers and outputting, an indication of whether the patient is infected with COVID-19. The method can detect COVID-19 early and rapidly from chest X-ray images in presence of other lung diseases including viral/bacterial pneumonia and is robust to different severity levels of the diseases.

### STATEMENT REGARDING PRIOR DISCLOSURE BY THE INVENTORS

Aspects of this technology are described in an article Tamal M, Alshammari M, Alabdullah M, Hourani R, Alola H A, Hegazi T M. An integrated framework with machine learning and radiomics for accurate and rapid early diagnosis of COVID-19 from Chest X-ray. Expert Syst Appl. 2021 Oct. 15; 180:115152. doi: 10.1016/j.eswa.2021.115152. Epub 2021 May 4. PMID: 33967406; PMCID: PMC8095015, which is herein incorporated by reference in its entirety.

### BACKGROUND

### Technical Field

The present disclosure is directed to a system, method, and computer readable storage medium that integrates chest x-ray and machine learning for diagnosis of COVID-19, and in particular uses radiomics features and ensemble bagged model trees for distinguishing COVID-19 from other lung diseases including bacterial pneumonia and viral pneumonia, as well as SARS, especially at very early stages.

### Related Art

COVID-19 (more formally, 2019 novel coronavirus, SARS-Cov-2) is currently a major health crisis that the world is experiencing. See Chan, J.-F.-W., Yuan, S., Kok, K.-H., To, K.-K.-W., Chu, H., Yang, J., . . . Yuen, K.-Y. (2020). A familial cluster of pneumonia associated with the 2019 novel coronavirus indicating person-to-person transmission: A study of a family cluster. The Lancet, 395, 514-523; and Zhu, N., Zhang, D., Wang, W., Li, X., Yang, B., Song, J., . . . Tan, W. (2020). A Novel Coronavirus from Patients with Pneumonia in China, 2019. New England Journal of Medicine, 382, 727-733, each incorporated herein by reference in their entirety. According to the World Health Organization (WHO) the disease is highly infectious and around 1 out of every 5 people who gets COVID-19 caused by 2019 novel coronavirus (2019-nCoV) needs to be hospitalized due to breathing difficulty. See WHO. (2020). Media Statement: Knowing the risks for COVID-19, incorporated herein by reference in its entirety. To avoid burden on the healthcare system and to reduce the spread of the disease, it is vital to carry out fast and accurate diagnosis of COVID-19. In clinical practice, real time polymerase chain reaction (RT-PCR) is considered as gold standard in COVID-19 diagnosis. Though the specificity of RT-PCR test is high, the sensitivity varies between 71 to 98% depending on sample collection site and sample quality, stage of disease and degree of viral multiplication or clearance (Wolfel et al., 2020). See Bwire, G. M., Majigo, M. V., Njiro, B. J., & Mawazo, A. (2020). Detection profile of SARSCoV-2 using RT-PCR in different types of clinical specimens: A systematic review and meta-analysis. J Med Virol; Wang, W., Xu, Y., Gao, R., Lu, R., Han, K., Wu, G., & Tan, W. (2020). Detection of SARSCoV-2 in Different Types of Clinical Specimens. JAMA; Watson, J., Whiting, P. F., & Brush, J. E. (2020). Interpreting a covid-19 test result. BMJ, 369, Article m1808; Sethuraman, N., Jeremiah, S. S., & Ryo, A. (2020). Interpreting Diagnostic Tests for SARS-CoV-2. JAMA; and Wolfel, R., Corman, V. M., Guggemos, W., Seilmaier, M., Zange, S., Muller, M. A., . . . Wendtner, C. (2020). Virological assessment of hospitalized patients with COVID-2019. Nature, 581, 465-469, each incorporated herein by reference in their entirety. Other different types of samples (e.g., blood, urine, stool etc.) were also used for COVID-19 detection with variable results. See Xiaolong, C. (2020). Landscape Coronavirus Disease 2019 test (COVID-19 test) in vitro—A comparison of PCR vs Immunoassay vs Crispr-Based test, incorporated herein by reference in its entirety.

It has been reported that chest CT demonstrates higher sensitivity for the diagnosis of COVID-19 compared to initial RT-PCR tests of pharyngeal swab samples. See Ahmed, S., Yap, M. H., Tan, M., & Hasan, M. K. (2020). ReCoNet: Multi-level Preprocessing of Chest X-rays for COVID-19 Detection Using Convolutional Neural Networks. Preprint from medRxiv, 2020.2007.2011.20149112; and Hu, Q., Guan, H., Sun, Z., Huang, L., Chen, C., Ai, T., . . . Xia, L. (2020). Early CT features and temporal lung changes in COVID-19 pneumonia in Wuhan. China. European Journal of Radiology, 128, Article 109017, each incorporated herein by reference in their entirety. Another study pointed out that chest CT scans suggested presence of COVID-19 related pneumonia for 81% of the patients who were tested negative with RT-PCR. See Ai, T., Yang, Z., Hou, H., Zhan, C., Chen, C., Lv, W., . . . Xia, L. (2020). Correlation of Chest CT and RT-PCR Testing for Coronavirus Disease 2019 (COVID-19) in China: A Report of 1014 Cases. Radiology, 296, E32-E40, incorporated herein by reference in its entirety. According to the Radiological Society of North America (RSNA), the sensitivity of CT to detect COVID-19 infection was 98% compared to RT-PCR sensitivity of 71%. See Fang, Y., Zhang, H., Xie, J., Lin, M., Ying, L., Pang, P., & Ji, W. (2020). Sensitivity of Chest CT for COVID-19: Comparison to RT-PCR. Radiology, 296, E115-E117, incorporated herein by reference in its entirety. Thus, CT could be considered as a primary tool for COVID-19 detection, unless the patient cannot be moved. See RSNA Press Release. (2020). CT Provides Best Diagnosis for COVID-19. In: RSNA, incorporated herein by reference in its entirety.

Chest X-ray (CXR) has also been considered, but has been found to be an insensitive method specifically in the detection of COVID-19 at early disease stage. CXR has lower spatial resolution compared to CT, and subsequently CXR has a reported low sensitivity of 59% for initial detection and may even appear normal until 4-5 days after the start of symptoms. See Guan, W. J., Ni, Z. Y., Hu, Y., Liang, W. H., Ou, C. Q., He, J. X., Liu, L., Shan, H., Lei, C. L., Hui, D. S. C., Du, B., Li, L. J., Zeng, G., Yuen, K. Y., Chen, R. C., Tang, C. L., Wang, T., Chen, P. Y., Xiang, J., Li, S. Y., Wang, J. L., Liang, Z. J., Peng, Y. X., Wei, L., Liu, Y., Hu, Y. H., Peng, P., Wang, J. M., Liu, J. Y., Chen, Z., Li, G., Zheng, Z. J., Qiu, S. Q., Luo, J., Ye, C. J., Zhu, S. Y., Zhong, N. S., & China Medical Treatment Expert Group for, C. (2020). Clinical Characteristics of Coronavirus Disease 2019 in China. N Engl J Med, 382, 1708-1720; and Wang, Y., Dong, C., Hu, Y., Li, C., Ren, Q., Zhang, X., . . . Zhou, M. (2020). Temporal Changes of CT Findings in 90 Patients with COVID-19 Pneumonia: A Longitudinal Study. Radiology, 296, E55-E64, each incorporated herein by reference in their entirety. However, because of availability and portability, CXR can be utilized as both baseline and follow up imaging method for monitoring disease progression. See Simpson, S., Kay, F. U., Abbara, S., Bhalla, S., Chung, J. H., Chung, M., Henry, T. S., Kanne, J. P., Kligerman, S., P., K., J., & Litt, H. (2020). Radiological Society of North America Expert Consensus Document on Reporting Chest CT Findings Related to COVID-19: Endorsed by the Society of Thoracic Radiology, the American College of Radiology, and RSNA. Radiology: Cardiothoracic Imaging, 2, e200152, incorporated herein by reference in its entirety. Furthermore, besides availability and portability, CXR has several advantages over other conventional COVID-19 diagnostic tests, e.g., short examination time, low cost and can be sterilized easily and quickly. See Sarkodie, B. D., Osei-Poku, K., & Brakohiapa, E. (2020). Diagnosing COVID-19 from Chest X-ray in Resource-Limited Environment-Case Report, Medical case report. In (pp. 3), incorporated herein by reference in its entirety.

Several deep neural network based machine learning approaches have been proposed to detect COVID-19 directly from CXR. The reported sensitivity and specificity vary from 85.35 to 96.7% and 90 to 100% respectively based on methods, disease types and classification of the diseases. See Ahmed et al., 2020; Asif, S., Wenhui, Y., Jin, H., Tao, Y., & Jinhai, S. (2020). Classification of COVID-19 from Chest X-ray images using Deep Convolutional Neural Networks. Preprint from medRxiv; Basu, S., Mitra, S., & Saha, N. (2020). Deep Learning for Screening COVID-19 using Chest X-Ray Images. Preprint from medRxiv; Chowdhury, M. E. H., Rahman, T., Khandakar, A., Mazhar, R., Kadir, M. A., Mahbub, Z., Islam, K. R., Khan, M. S., Al-Emadi, N., & Reaz, M. B. I. (2020). Can AI help in screening Viral and COVID-19 pneumonia? Preprint form arvix; and Ozturk, T., Talo, M., Yildirim, E. A., Baloglu, U. B., Yildirim, O., & Rajendra Acharya, U. (2020). Automated detection of COVID-19 cases using deep neural networks with X-ray images. Computers in Biology and Medicine, 121, Article 103792, each incorporated herein by reference in their entirety. The detail of each method is provided in Table 1. Because of the extensive number of hidden layers and large number of connections between nodes, deep learning neural network based machine learning approaches require a large number of images to train. To obviate the challenges of limited CXR COVID-19 data, transfer learning along with data augmentation has been implemented which allows an increase in the diversity of the data. However, it is to be noted that data augmentation can only increase the data via geometric transformation of the images and cannot increase the number of images with different COVID-19 conditions.

TABLE 1
CXR based COVID-19 detection literature

| Name of | | | | Data Description |
|---|---|---|---|---|
| the paper | Accuracy | sensitivity | specificity | and Classes |
| Covallo et al. | 91.8% | 93% | 90% | positive/negative |
| Texture Analysis in the | | | | |
| Evaluation of Covid-19 | | | | |
| Pneumonia in Chest X-Ray | | | | |
| Images: a Proof of Concept | | | | |
| Study | | | | |
| Asif et al. | 98% | NA | NA | Normal 1341, Viral |
| Classification of | (training accuracy | | | Pneumonia 1345, |
| COVI0 -19 from Chest X-ray | of 97% and | | | COVI0 -19 864 (data |
| images using Deep | validation | | | augmentation and |
| Convolutional Neural | accuracy of 93%). | | | splitting for training, |
| Networks Sohaib Asif, Yi | | | | validation and test) |
| Wenhui*, Hou Jin, Yi Tao, | | | | |
| Basu et al. | 95.3% ± 0.02 | NA | NA | Normal, Pneumonia, |
| Deep Learning for Screening | overall, 100% of | | | COVI0 -1, other disease |
| COVID-19 using Chest X-Ray | the covid and | | | (atelectasis, |
| Images | normal cases | | | cardiomegaly, |
| | being correctly | | | infiltration, effusion, |
| | classified in each | | | nodule, mass) |
| | validation fold | | | 225 Covid-19 108,948 |
| | | | | frontal chest x-ray |
| | | | | 5-fold cross validation |
| Ahmed 2020 et al | 97.48% | 96.39% | 97.53% | Training: |
| ReCoNet - Multilevel | | | | COVI0-19 207, Normal |
| Preprocessing of Chest | | | | 7966, Pneumonia 5451 |
| X-rays for COVID-19 | | | | Test: |
| Detection Using | | | | COVI0 -19 31, Normal |
| Convolutional Neural | | | | 885, Pneumonia 594 |
| Networks | | | | |
| Chowdhury 2020 | Normal vs Covid-19 | Normal vs Covid-19 | Normal vs Covid-19 | Training: |
| Can AI help in screening | 98.3% | 96.7% | 100% | COVI0 -19 130, Normal |
| Viral and COVI0 -19 | Normal vs. viral | Normal vs viral | Normal vs viral | 190, Viral Pneumonia |
| pneumonia | vs. Covid-19 | vs Covid-19 | vs Covid-19 | 190 |
| | 98.3% | 96.7% | 99% | Test: |
| | | | | COVI0 -19 60, Normal |
| | | | | 1151, Viral Pneumonia |
| | | | | 594 |
| Ozturk et al. | 98.08% | 95.3 | 95.3 | binaryclass classification |
| Automated detection of | | | | (COVID vs. No-Findings |
| COVID- 19 cases using deep | 87.02% | 85.35 | 92.18 | Multiclass |
| neural networks with X-ray | | | | (COVID vs. No-Findings |
| images | | | | vs Pneumomia) |

| Name of the paper | Algorithm Description | Models |
|---|---|---|
| Covallo et al. | After samples separation | Ensemble Machine |
| Texture Analysis in the | into training (n = 132) | Learning (cut-off |
| Evaluation of Covid-19 | and test (n = 88) sets | 132.57) |
| Pneumonia in Chest X-Ray | {60:40 ratio), the | |
| Images: a Proof of Concept | training test was used to | |
| Study | train 11 classification | |
| | models: Partial Least | |
| | Square Discriminant | |
| | Analysis {PLS-DA), Naive | |
| | Bayes {NB), Generalized | |
| | Linear Model | |
| | {GLM),Logistic Regression | |
| | {LR), Fast Large Margin | |
| | {FM L), Deep Learning | |
| | {DL), Decision Tree {OT), | |
| | Random Forest (RF), | |
| | Gradient Boosted Trees | |
| | {GBT), artificial Neural | |
| | Network (aNN) and Support | |
| | Vector Machine {SVM) | |
| Asif et al. | 88% for training, 7% for | based model Inception |
| Classification of | validation and 5% for | V3 with transfer |
| COVI0 -19 from Chest X-ray | testing | learning have been |
| images using Deep | | CNN |
| Convolutional Neural | | |
| Networks Sohaib Asif, Yi | | |
| Wenhui*, Hou Jin, Yi Tao, | | |
| Basu et al. | The adopted procedure is | CNN, Connected with |
| Deep Learning for Screening | outlined below. | Gradient Class |
| COVID-19 using Chest X-Ray | Instantiate the | Activation Map |
| Images | convolutional base of the | {GradCAM) for |
| | model trained on Data-A | detecting the regions |
| | and load its pretrained | Domain extension |
| | weights. | transfer learning |
| | Replace the last fully | {DETL) |
| | connected layer of the | |
| | pre-trained CNN with a | |
| | new fully connected | |
| | layer. | |
| | Freeze the layers of the | |
| | model up to the last | |
| | convolutional block. | |
| | Finally retrain the last | |
| | convolution block and the | |
| | fully connected layers | |
| | using Stochastic Gradient | |
| | Descent {SGD) | |
| | optimization algorithm | |
| | with a very slow learning | |
| | rate. | |
| Ahmed 2020 et al | Used 90% of the data for | ReCoNet - preprocessing |
| ReCoNet - Multilevel | training and 10% for | before feeding to |
| Preprocessing of Chest | validation. | deep net in the same |
| X-rays for COVID-19 | | framework |
| Detection Using | | |
| Convolutional Neural | | |
| Networks | | |
| Chowdhury 2020 | | Four different network: |
| Can AI help in screening | | 1) ALexNet, 2) ResNet28, |
| Viral and COVID-19 | | 3) DenseNet201 and |
| pneumonia | | 4) SqueezeNet |
| Ozturk et al. | 80% of X-ray images are | Dark CovidNet |
| Automated detection of | used for training and 20% | architecture |
| COVID- 19 cases using deep | for validation. | |
| neural networks with X-ray | | |
| images | | |

On the other hand, machine learning techniques that depend on handcrafted features extraction and selection approaches can be trained with a smaller data set. In depth analysis of different first, second and third order statistical features known as radiomics have been successfully utilized for decoding the radiographic phenotype in cancer. See Aerts, H. J., Velazquez, E. R., Leijenaar, R. T., Parmar, C., Grossmann, P., Carvalho, S., . . . Lambin, P. (2014). Decoding tumour phenotype by noninvasive imaging using a quantitative radiomics approach. Nat Commun, 5, 4006; and van Griethuysen, J. J. M., Fedorov, A., Parmar, C., Hosny, A., Aucoin, N., Narayan, V., . . . Aerts, H. J. W. L. (2017). Computational Radiomics System to Decode the Radiographic Phenotype. Cancer Research, 77, Article e104, each incorporated herein by reference in their entirety. Recent studies prove the potential of differentiating glioblastomas (GBM) from metastatic brain tumors (MBTs) on contrast-enhanced T1 weighted imaging with radiomics based a machine learning method. See Chen, C., Ou, X., Wang, J., Guo, W., & Ma, X. (2019). Radiomics-Based Machine Learning in Differentiation Between Glioblastoma and Metastatic Brain Tumors. Frontiers. Oncology, 9, incorporated herein by reference in its entirety. However, only one study so far has reported achieving 93% sensitivity and 90% specificity in detecting COVID-19 by applying different machine learning algorithms on textural features extracted from CXR. See Cavallo, A. U., Troisi, J., Forcina, M., Mari, P., Forte, V., Sperandio, M., Pagano, S., Cavallo, P., Floris, R., & Geraci, F. (2020). Texture Analysis in the Evaluation of Covid-19 Pneumonia in Chest X-Ray Images: a Proof of Concept Study. Preprint from Research Square, 25 Jun. 2020, incorporated herein by reference in its entirety.

The training and performance evaluation of most of these methods were carried out by randomly splitting the data into training, test and validation sets. The sensitivity and specificity of these methods were evaluated without consideration of level of severity of disease. Applicant has determined that the performance of a diagnosis method is very much dependent on the level of disease severity. In addition, applicant has determined that performance evaluation should be assessed on an independent data set in order to be successful in the field, where the independent data set is a data set that is acquired for a different set of patients than patient data that is used for training.

### SUMMARY

An aspect of the present disclosure relates to a method for diagnosis of COVID-19 from at least one Chest X-Ray image. The method can include imaging, by a chest x-ray machine, a person's chest area to obtain the at least one chest x-ray image; performing, by processing circuitry, image segmentation of a human lung in the at least one Chest X-Ray image; extracting, by the processing circuitry, radiomics features from the segmented lung; selecting, by the processing circuitry, a subset of the radiomics features for classification ability between two classes of COVID-19 and non-COVID-19 including other lung diseases, wherein the subset of radiomics features includes: 13 first order, 3 2D shape based, 20 Gray Level Co-occurrence Matrix (GLCM), 8 Gray Level Departure Matrix (GLDM), 10 Gray Level Run Length Matrix (GLRLM), 13 Gray Level Size Zone Matrix (GLSZM) and 4 Neighboring Gray Tone Difference Matrix (NGTDM) features; classifying, by the processing circuitry, between COVID-19 and non-COVID-19 using an ensemble bagged model having a plurality of classifiers; and outputting, by the processing circuitry, an indication of whether the patient is infected with COVID-19.

A further aspect of the present disclosure relates to a mobile device for diagnosis of COVID-19 from at least one Chest X-Ray image. The mobile device can include a display device; and processing circuitry. The processing circuitry is configured to perform image segmentation of a human lung in the at least one Chest X-Ray image; extract radiomics features from the segmented lung; select a subset of the radiomics features for classification ability between two classes of COVID-19 and non-COVID-19 including other lung diseases, wherein the subset of radiomics features includes: 13 first order, 3 2D shape based, 20 Gray Level Co-occurrence Matrix (GLCM), 8 Gray Level Departure Matrix (GLDM), 10 Gray Level Run Length Matrix (GLRLM), 13 Gray Level Size Zone Matrix (GLSZM) and 4 Neighboring Gray Tone Difference Matrix (NGTDM) features; classify between COVID-19 and non-COVID-19 using an ensemble bagged model having a plurality of classifiers; and output to the display device an indication of whether the patient is infected with COVID-19.

A further aspect of the present disclosure relates to a non-transitory computer readable storage medium storing program instructions, which when executed by processing circuitry performs a method for diagnosis of COVID-19 from at least one Chest X-Ray image. The method can include performing image segmentation of a human lung in the at least one Chest X-Ray image; extracting radiomics features from the segmented lung; selecting a subset of the radiomics features for classification ability between two classes of COVID-19 and non-COVID-19 including other lung diseases, wherein the subset of radiomics features includes: 13 first order, 3 2D shape based, 20 Gray Level Co-occurrence Matrix (GLCM), 8 Gray Level Departure Matrix (GLDM), 10 Gray Level Run Length Matrix (GLRLM), 13 Gray Level Size Zone Matrix (GLSZM) and 4 Neighboring Gray Tone Difference Matrix (NGTDM) features; classifying between COVID-19 and non-COVID-19 using an ensemble bagged model having a plurality of classifiers; and outputting an indication of whether the patient is infected with COVID-19.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1 is a schematic diagram of a chest x-ray (CXR) system in accordance with an exemplary aspect of the disclosure;

FIG. 2 is a block diagram of a computing device for the chest x-ray system;

FIG. 3 is a block diagram of a dedicated computing device that is an integral component of the CXR apparatus in accordance with an exemplary aspect of the disclosure;

FIG. 4 illustrates the method for COVID-19 detection from C×R images in accordance with an exemplary aspect of the disclosure;

FIG. 5 is flow diagram for an ensemble bagged model for the method for COVID-19 detection;

FIGS. 6A, 6B, and 6C illustrate normal CXR (a) and RT-PCR test confirmed COVID-19 positive cases with high (b) and low (c) severity score;

FIG. 7 illustrates the Z-score heatmap of 71 radiomics features that yield statistically significant difference between COVID-19 and other lung diseases, wherein each row represents one feature and each column represents one C×R image used in the training set;

FIGS. 8A and 8B are graphs illustrating the ROC plot of the three best performing classifiers during training (left) and during testing (right); and

FIG. 9 illustrates the sensitivity and specificity for whole test data as well as for each severity level, wherein the size of the filled circles represents severity levels from 0 to 4.

### DETAILED DESCRIPTION

Although researchers have been moving forward with efforts to create a computer-aided method and device that utilizes vision (optically enhanced images) that recognize COVID-19 in medical imagery, the Centers for Disease Control and Prevention (CDC) currently does not recommend the use of CT scans (Computed tomography) or X-rays for COVID-19 diagnosis. A CT scan is an X-ray image made using a form of tomography in which a computer controls the motion of the X-ray source and detectors, processes the data, and produces the image. In addition, the American College of Radiology (ACR) and similar radiological organizations in Canada, New Zealand, and Australia have also released statements telling radiologists that they do not currently recommend the use of CT scans for COVID-19 detection.

The American College of Radiology (ACR) Thoracic Imaging Panel noted difficulties distinguishing the difference between COVID-19 and common lung infections like bacterial or viral pneumonia. ACR recommends that even if a chest X-ray exhibits signs of COVID-19, a lab test is still required for confirmation. On the other hand, despite this position, ACR considers AI for COVID-19 detection from CT scans a potentially useful tool in health care settings where there are few human radiologists available. In any case, there is a need to distinguish between other illnesses that appear in CT or X-ray imagery, especially in early stages where even a radiologist may not be able to detect signs of COVID-19 in a chest x-ray.

COVID-19 is a worldwide pandemic, where there are shortages of radiologists in certain regions. Rapid and early diagnosis of COVID-19 is needed for the health benefit of people exhibiting no signs, as well as those who may have contracted the virus, or people exhibiting COVID-like symptoms but due to other lung diseases. In addition, there is a need to monitor variants of COVID-19. These variants seem to spread more easily and quickly than other variants, which may lead to more cases of COVID-19. An increase in the number of cases will put more strain on healthcare resources, lead to more hospitalizations, and potentially more deaths.

There is a need for a diagnosis method for COVID-19 that is robust against different severity levels and that can distinguish variants of COVID-19 over other lung diseases that may have similar symptoms. A solution is a radiomics based machine learning approach to detect COVID-19 from CXR that is robust for all levels of severity. The approach includes selection strategy to pick the most suitable features for binary classification for various levels of severity. The sensitivity and specificity of the disclosed approach is verified on a completely independent test data set (i.e., different set of patients) containing normal, viral and bacterial pneumonia and confirmed COVID-19 with different levels of severity determined by two experienced radiologists.

There is a need for an efficient and rapid early diagnosis capability of COVID-19 detection, especially in heavily populated areas that require diagnosis in a wide scale, or in events, such as sporting events or large conferences, that may require fast, efficient diagnosis of a large number of participants and/or attendees.

### Chest X-Ray System

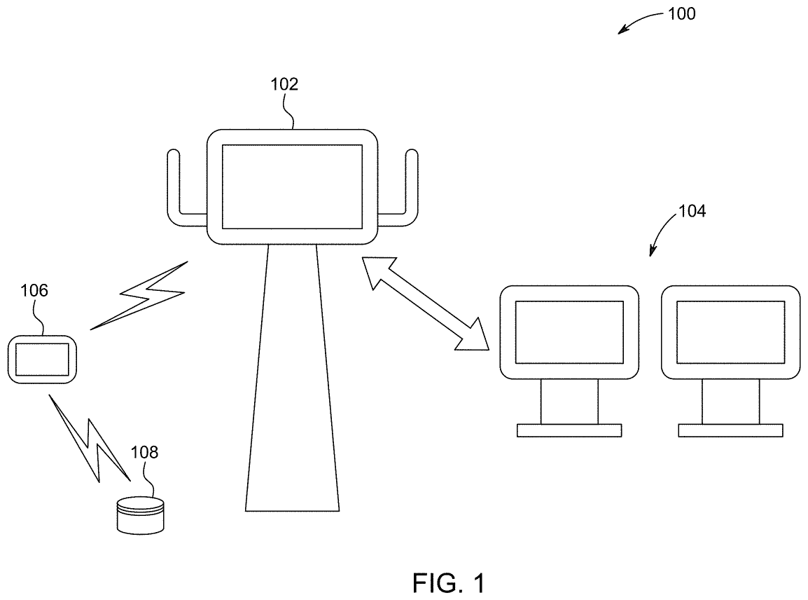

FIG. 1 is a schematic diagram of a chest x-ray (CXR) system in accordance with an exemplary aspect of the disclosure. The equipment typically used for chest x-rays consists of a wall-mounted or stand-mounted, box-like CXR apparatus 102 containing the x-ray film, or a special plate that records the image digitally. An x-ray producing tube is typically positioned about six feet away.

The CXR apparatus 102 may also be arranged with the x-ray tube suspended over a table on which the patient lies. A drawer under the table holds the x-ray film or digital recording plate.

A portable x-ray machine is a compact apparatus that can be taken to the patient in a hospital bed or the emergency room. The x-ray tube is connected to a flexible arm that is extended over the patient while an x-ray film holder or image recording plate is placed beneath the patient.

On a chest x-ray, the ribs and spine will absorb much of the radiation and appear white or light gray on the image. Lung tissue absorbs little radiation and will appear dark on the image. Most x-ray images are digital files that are stored electronically. These stored images are easily accessible for diagnosis and disease management.

The C×R system 100 may include one or more display monitors or image workstations 104 to display C×R images obtained by the apparatus 102 and perform image processing functions, such as zoom, crop, brightening, contrast enhancement, and other image editing functions.

Disclosed embodiments provide an efficient and rapid early diagnosis capability of COVID-19 detection. An aspect is an efficient machine learning model that can be performed efficiently and accurately in a mobile device. Performing the machine learning in a mobile device enables COVID-19 diagnosis in heavily populated areas that require diagnosis in a wide scale, or in events, such as sporting events or large conferences, that may require fast, efficient diagnosis of a large number of participants and/or attendees. In these embodiments, the C×R system 100 may include several CXR apparatuses 102. A computing device 106 may communicate with the CXR apparatuses 102 to performing COVID-19 detection. In some embodiments, the computing device 106 may be a mobile device, such as a smartphone, tablet computer, or laptop computer. The mobile device may include a mobile application (App) for performing COVID-19 detection. The mobile application may store data related to the COVID-19 detection in a database system 108, which may be a central database system for the C×R system 100.

In some embodiments, the COVID-19 detection may be performed in the workstation 104, without the computing device 106. In some embodiments, the computing device 106 may be an integral component of a CXR apparatus 102.

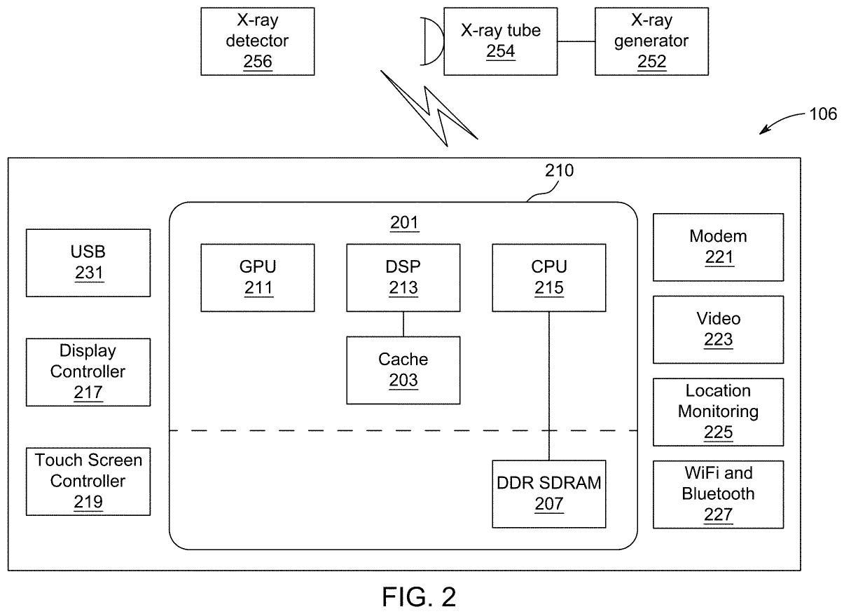

FIG. 2 is a block diagram of a computing device for the chest x-ray system. As mentioned above, the computing device 106 may be a mobile device that performs a mobile application (App) to be used in conjunction with the chest x-ray apparatus 102, or several chest x-ray apparatuses. The X-ray apparatus 102 is configured with an X-ray detector 256, an X-ray generator 252, an X-ray tube 254. The mobile device may provide support for various functions such as simultaneous camera sensor inputs, video decoding and playback, location services, wireless communications, and cellular services. The computing device 106 contains processing circuitry that includes a central processing unit (CPU) 215, and may also include special purpose processors such as a graphics processing unit (GPU) 211 and a digital signal processor (DSP) 213. The CPU 215 may include a memory storing executable program instructions for COVID-19 detection, where the memory may be any of several types of volatile memory 207, including RAM, SDRAM, DDR SDRAM, to name a few. The CPU 215 outputs a timing signal for the X-ray tube 254 to irradiate X-ray to the X-ray generator 252, inputs a chest X-ray moving image from the X-ray detector 256. The DSP 213 may include one or more dedicated caches 203 in order to perform computer vision functions as well as machine learning functions. The GPU 211 performs graphics processing for a high resolution display device, e.g., 4K or greater. The GPU 211, DSP 213, CPU 215, Cache 203, and in some embodiments, a cellular modem 221, may all be contained in a single system-on-chip (SOC) 201. The computing device 106 may also include video processing circuitry 223 for video decoding and playback, location service circuitry 225, including GPS and dead reckoning, and connectivity service circuitry 227, including WiFi and Bluetooth. The computing device 106 may include one or more input/output ports, including USB connector(s) 231, such as connectors for USB 2, USB 3, and further enhancements thereof.

A mobile device as the computing device 106 may include a display device, such as a touch screen display device. The display device is controlled by a display controller 217, while the touch screen functions are processed by a touch screen controller 219. In one embodiment, the connectivity service circuitry 227 may be configured to wirelessly communicate with a plurality of CXR apparatuses 102 to receive respective chest x-ray images for several patients. The mobile device 106 performs instructions for COVID-19 detection and outputs to the touch screen display device, while simultaneously storing in the database system 108, an indication for each of the plurality of patients whether the respective patient is infected with COVID-19. The indication may be a simple display of a patient name together with text that states the patient's status as a result of COVID-19 detection. In one embodiment, the indication may be the patient's name or other id together with the chest x-ray image for the patient, or a link to the chest x-ray image which may be stored in the database system 108 or other external storage for chest x-rays.

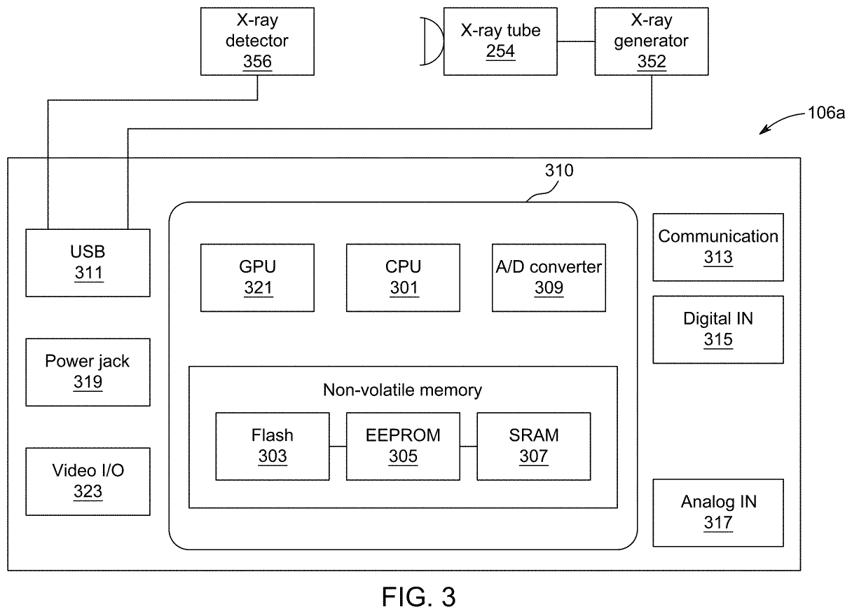

FIG. 3 is a block diagram of a dedicated computing device that is an integral component of the CXR apparatus in accordance with an exemplary aspect of the disclosure. The integral computing device 106a may be based on an embedded microcontroller. An embedded microcontroller may contain one or more processor cores (CPUs) along with memory (volatile and non-volatile) and programmable input/output peripherals. Program memory in the form of flash, ROM, EPROM, or EEPROM may be included on chip, as well as a secondary RAM for data storage. The X-ray apparatus 102 is configured with an X-ray detector 356, an X-ray generator 352, an X-ray tube 354. The integral computing device 106a may provide support for various functions such as simultaneous camera sensor inputs, and video decoding and playback. In one embodiment, the integral computing device 106a is an integrated circuit board having a microcontroller 310. The microcontroller 310 outputs a timing signal for the X-ray tube 354 to irradiate X-ray to the X-ray generator 352, inputs a chest X-ray moving image from the X-ray detector 356. The board includes digital I/O pins 315, analog inputs 317, hardware serial ports 313, a USB connection 311, a power jack 319, and a video I/O port 323. Although the dedicated computing device 106a of FIG. 3 is a typical microcontroller-based board, it should be understood that other microcontroller board configurations are possible. Variations can include the number of pins, whether or not the board includes communication ports or a reset button. Microcontrollers may vary based on the number of processing cores, size of non-volatile memory, the size of data memory, as well as whether or not it includes an A/D converter or D/A converter.

### Materials and Methods

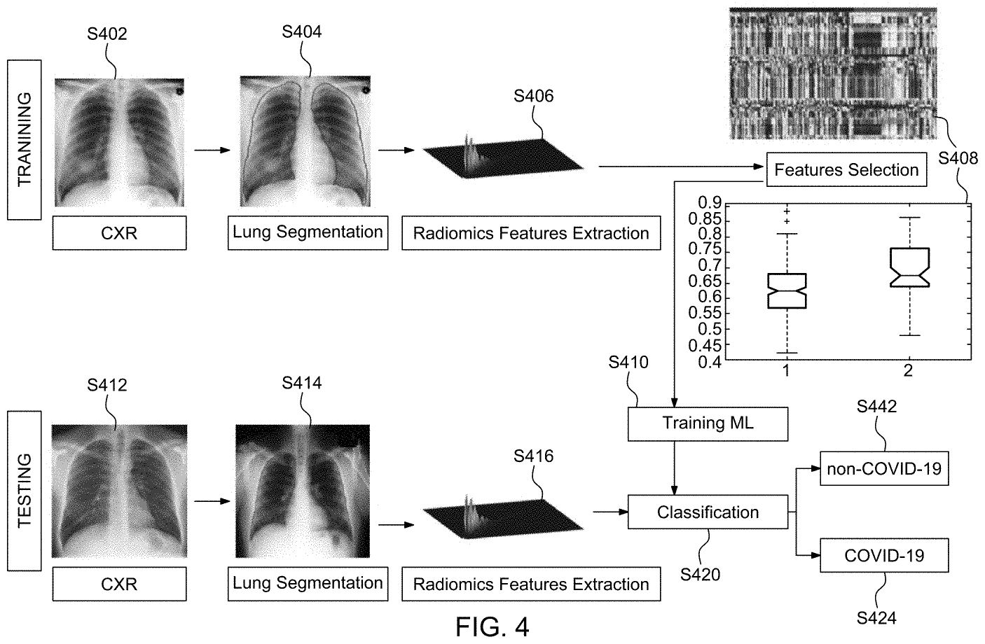

FIG. 4 illustrates the method for COVID-19 detection from C×R images in accordance with an exemplary aspect of the disclosure.

Training Data Set and Radiomics Features Extraction

In one or more embodiments, in S402, each lung image is first manually delineated from a training C×R image set. In one embodiment, image smoothing or other processing technique is not applied before radiomics features extraction. The training dataset may be created from publically available data repositories containing normal and different types of lung conditions. Since an objective is to detect COVID-19 from other lung conditions, all C×R images may be grouped into two classes (COVID-19 and non-COVID-19). The details of the training data are provided in Table 1.

TABLE 1
Training data description

| Disease | | No of | |
|---|---|---|---|
| Type | Description | CXR | Source |
| non-COVID-19 | Normal | 50 | JSRT (Shiraishi |
| (Total 152 | | | et al., 2000) |
| images) | Viral/bacterial | 50 | Kaggle (Kaggle, |
| | pneumonia | | 2020) |
| | Other lung conditions | 52 | GitHub (Github, |
| | (ARDS (4), SARS (15), | | 2020) |
| | Pneumocystis (12), | | |
| | Streptococcus (13), | | |
| | Chlamydophila | | |
| | (1), E. Coli (4), | | |
| | Klebsiella (1) | | |
| | and Legionella (1)) | | |
| COVID-19 | COVID-19 | 226 | GitHub (Github, |
| | | | 2020) |

To avoid bias in class distribution for training, Adaptive Synthetic (ADASYN) oversampling approach may be implemented on the non COVID-19 dataset to balance the imbalanced dataset. ADASYN synthetically creates new samples in between difficult-to-classify samples from the minority class. See Haibo, H., Yang, B., Garcia, E. A., & Shutao, L. (2008). ADASYN: Adaptive synthetic sampling approach for imbalanced learning. In In 2008 IEEE International Joint Conference on Neural Networks (IEEE World Congress on Computational Intelligence) (pp. 1322-1328), incorporated herein by reference in its entirety. In S404, segmentation of a lung on all the C×R images except the normal ones is performed using a tool such as MATLAB 2019b Image Segmenter app. See Brown, M. S., Wilson, L. S., Doust, B. D., Gill, R. W., & Sun, C. (1998). Knowledge-based method for segmentation and analysis of lung boundaries in chest X-ray images. Computerized Medical Imaging and Graphics, 22, 463-477, incorporated herein by reference in its entirety. For the normal cases, publically available lung masks may be used. In S406, approximately 100 radiomics features are then extracted using the segmented lung and a tool such as PyRadiomics tool with Python 3.7.6 for each lung separately (van Griethuysen et al., 2017). This extraction may yield 18 first-order statistics, 9 2D shape-based, 22 Gray Level Co-occurrence Matrix (GLCM), 16 Gray Level Run Length Matrix (GLRLM), 16 Gray Level Size Zone Matrix (GLSZM), 5 Neighboring Gray Tone Difference Matrix (NGTDM) and 14 Gray Level Dependence Matrix (GLDM) features.

Among the extracted features, first order features describe the distribution of voxel intensities within the ROI. Shape based features represent 2D size and shape of the ROI, e.g., perimeter, elongation, sphericity etc. The remaining five feature matrices represent textural appearance. GLCM records the probability of occurrence of a pixel pair. The number of connected voxels within a distance δ that are dependent on the center voxel is used to generate GLDM. GLRLM is defined as the length in number of pixels having the same gray level value. On the other hand, GLSZM defines a zone of connected voxels with the same gray level intensity. GLSZM quantifies gray level zones within a ROI in an image. The number of connected voxels that share the same gray level intensity defines a gray level zone. NGTDM quantifies the difference between a gray value and the average gray value of its neighbors within distance δ.

Radiomics Features Selection

In S408, the features that have better classification ability between COVID-19 and other lung diseases are selected. Two methods of feature selection were implemented. The first method is a heatmap of Z-scores to identify features that can classify these two groups. The second method is the one-way ANOVA test to find the features that have a statistically significant difference between the means of the two classes with the criteria p<0.05. It was found that only 71 features out of 100 radiomics features show statistically significant difference and therefore, only these 71 features were used for training the machine learning algorithms. Out of these 71 features 13 are first order, 3 2D shape based, 20 GLCM, 8 GLDM, 10 GLRLM, 13 GLSZM and 4 NGTDM extracted features.

Training of Machine Learning Algorithms

In S410, the selected features may be used to train a machine learning classification model. A number of conventional supervised and unsupervised machine learning algorithms were evaluated using the Classification Learner App of MATLAB 2019b. To optimize the parameter of the machine learning algorithms to classify between COVID-19 and other lung diseases, a 10 fold cross validation approach was implemented. See Refaeilzadeh, P., Tang, L., & Liu, H. (2009). Cross-Validation. In L. Liu & M. T. ÖZsu (Eds.), Encyclopedia of Database Systems (pp. 532-538). Boston, Mass.: Springer US, incorporated herein by reference in its entirety.

Area under receiver operating characteristic (AUC-ROC) curve as well as sensitivity, specificity, and accuracy were calculated to evaluate the ability of the classifier to discriminate the COVID-19 CXR cases from the other lung disease cases, as well as a CXR for a normal lung having no disease. Sensitivity, specificity and accuracy were calculated as:

$$Sensitivity = \frac{T P}{T P + F N}$$

$$Specificity = \frac{T N}{T N + F P}$$

$$Accuracy = \frac{T P + T N}{T P + F N + T N + F P}$$

where TP, TN, FP and FN refer to true positive, true negative, false positive and false negative respectively.

The three best performing classifiers during the training phase—1) fine Gaussian support vector machine (SVM), 2) fine k-nearest neighbor (KNN) and 3) ensemble bagged model (EBM) trees were chosen for further evaluation on the test data. Among different conventional machine learning algorithms, SVM is suitable for both linear and nonlinear binary classification tasks. It is also one of the most used automatic classifiers in healthcare. See Cervantes, J., Garcia-Lamont, F., Rodriguez-Mazahua, L., & Lopez, A. (2020). A comprehensive survey on support vector machine classification: Applications, challenges and trends. Neurocomputing, 408, 189-215; Huang, S., Cai, N., Pacheco, P. P., Narrandes, S., Wang, Y., & Xu, W. (2018). Applications of Support Vector Machine (SVM) Learning in Cancer Genomics. Cancer Genomics Proteomics, 15, 41-51; and Yu, W., Liu, T., Valdez, R., Gwinn, M., & Khoury, M. J. (2010). Application of support vector machine modeling for prediction of common diseases: The case of diabetes and pre-diabetes. BMC Med Inform Decis Mak, 10, 16, each incorporated herein by reference in their entirety. In comparison, KNN is a simpler technique that stores all existing instances and then classifies any new instance based on a user defined similarity measure. However, the performance of KNN is very much dependent on the size of training examples. See Thanh Noi, P., & Kappas, M. (2017). Comparison of Random Forest, k-Nearest Neighbor, and Support Vector Machine Classifiers for Land Cover Classification Using Sentinel-2 Imagery. Sensors, Basel, 18, incorporated herein by reference in its entirety. On the other hand, a EBM tree classifier may be used due to its stability and has found uses in many applications like credit card fraud detection. See Zareapoor, M., & Shamsolmoali, P. (2015). Application of Credit Card Fraud Detection: Based on Bagging Ensemble Classifier. Procedia Computer Science, 48, 679-685, incorporated herein by reference in its entirety.

Ensemble Bagged Model

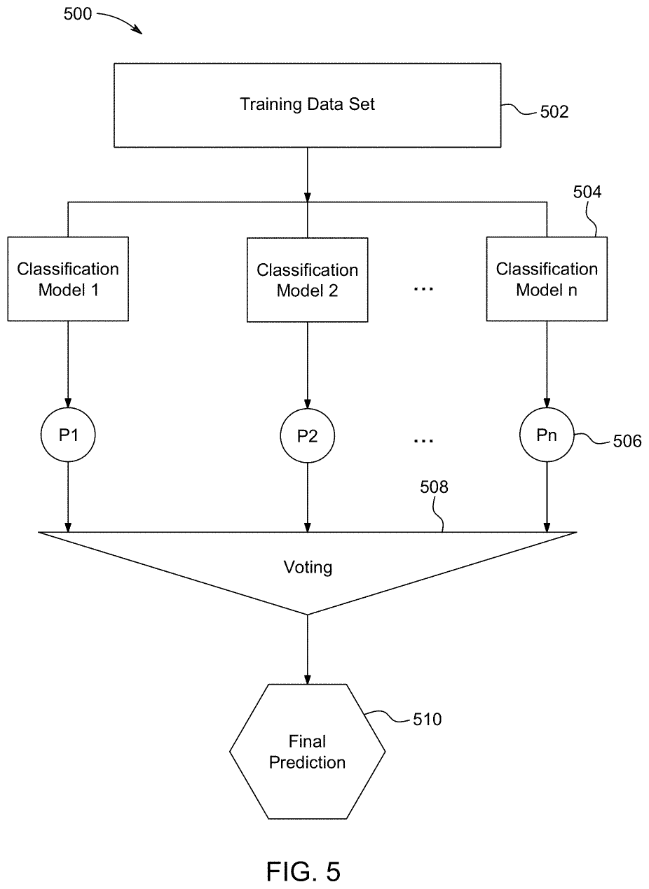

FIG. 5 is a flow diagram for an ensemble bagged model for the method for COVID-19 detection. The EBM 500 combines classifiers Classification Model 1, Classification Model 2, . . . Classification Model n, 504 into a meta-classifier. The classifiers 504 may be different classification algorithms, for example, decision trees, support vector machines, logistic regression classifiers, and K-nearest neighbor classifiers. Alternatively, the classifiers 504 may be the same base classification algorithm, training each classifier 504 with different subsets of the training dataset. In one embodiment, the EBM 500 may use the bagging algorithm, which combines several decision tree classifiers. The predictions P1, P2, . . . Pn, 506 of the classifiers 504 and used to determine the final classification prediction 510. In one embodiment, the EBM 500 may use a majority voting principle 508, in which a class is selected that has been predicted by a majority of the classifiers 504.

The EBM 500 may generate training instances for each of the classifiers 504. Bagging in the Ensemble Bagged Model is an approach to ensemble training in which a sample from a training data set is taken randomly with replacement, meaning that individual data points can be chosen more than once. In one embodiment, the training instances are sampled randomly with replacement from the training data set 502 in each round of bagging. In one embodiment, each sample is used to train a classifier 504.

Each classifier 504 may be trained using a decision tree learning algorithm. In one embodiment, the J48 Java implementation of the C4.5 decision tree algorithm is used as the classifier. The number of decision trees can be found using cross-validation.

In an exemplary implementation, the ensemble method is Random Forest Bagging, with Decision Tree classifiers. The exemplary ensemble method includes 30 bagged decision trees with a maximum of 680 decision splits. The depth of the decision tree classifier depends on other parameters (for example, Maximum number of decision splits, Minimum number of leaf node observations, and Minimum number of branch node observations). In each decision tree of the forest, 7 predictors are randomly selected for each split. For maximum depth, the default values of the tree depth controllers for bagging trees are: 680 maximum number of decision splits, 1 for minimum number of leaf node observations, and 2 for minimum number of branch node observations. The splitting criteria is Gini's diversity index.

Test Data Set

Once a machine learning model is trained (S410), it may be tested with a test data set. Often a test data set is obtained from the original training data set. For example, a training data set may be divided into 70% for training and 30% for testing. In disclosed embodiments, an independent data set is used for testing, where the independent data set is acquired from patients that are different from patients used for the training data set. In one embodiment, a Test CXR data set is the independent data set and consists of 165 C×R images (330 lungs) containing 25 normal, 25 viral/bacterial pneumonia and 115 COVID-19 cases. The 115 COVID-19 C×R images of 25 patients were acquired at local hospital. COVID-19 was confirmed with standard RT-PCR test. The details of the test data is shown in Table 2.

TABLE 2
Test data description

| Disease | | No of | |
|---|---|---|---|
| Type | Description | CXR | Source |
| non-COVID-19 | Normal | 25 | JSRT (Shiraishi |
| | | | et al., 2000 |
| | Viral/bacterial | 25 | Kaggle (2020) |
| | Pneumonia | | |
| COVID-19 | COVID-19 | 115 (25 | Local Hospital |
| | | patients) | |

In S412, Multiple chest X-rays were taken for all the 25 patients, in S414, segmentation is performed to extract the lungs, in S416, radiomics features are extracted. Out of 25 patients 15 patients passed away and 10 patients were cured and released from the hospital. Different C×R images of the same patient were organized by level of severity. Even for the same CXR, the severity between the two lungs are sometimes different according to the visual assessment of the radiologists. To evaluate the robustness of machine learning algorithms, the severity of each lung of each CXR for both COVID-19 and viral/bacterial pneumonia was scored. All CXRs were performed in a frontal projection in a posteroanterior view if the patient was able to stand; otherwise, an anteroposterior view in the sitting or supine position was acquired. The CXRs were independently evaluated by two experienced radiologists. In case of discrepant interpretations, the findings were resolved by consensus.

The radiologists rated pulmonary parenchymal involvement on CXR using a semiquantitative severity score (score 0 to 4) depending on the visual assessment on the extent of involvement by ground glass opacities (GGO) (i.e., hazy opacity not obliterating bronchi and vessels) or consolidations (i.e., area of attenuation obscuring airways and vessels). If none of these patterns were seen, then the radiologists would select score 0 (clear lung). Score 1=<25%, score 2=25-50%, score 3=50-75% and score 4=>75% involvement. Severity score 0 or 1 corresponds to early stage of both COVID-19 and non-COVID-19 infections.

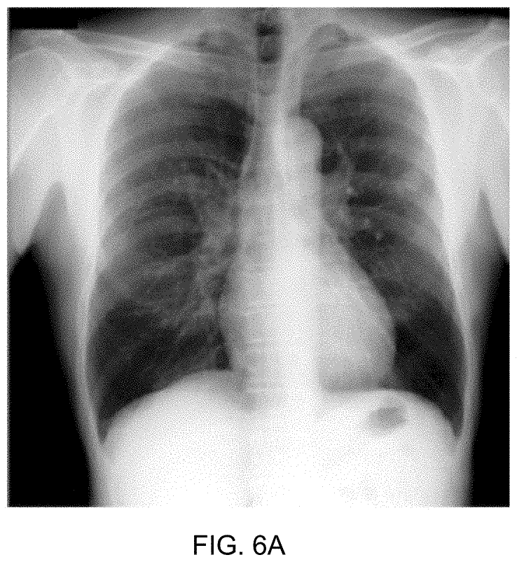

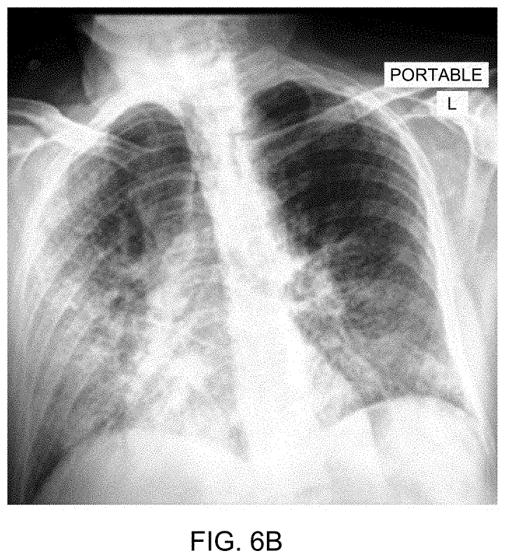

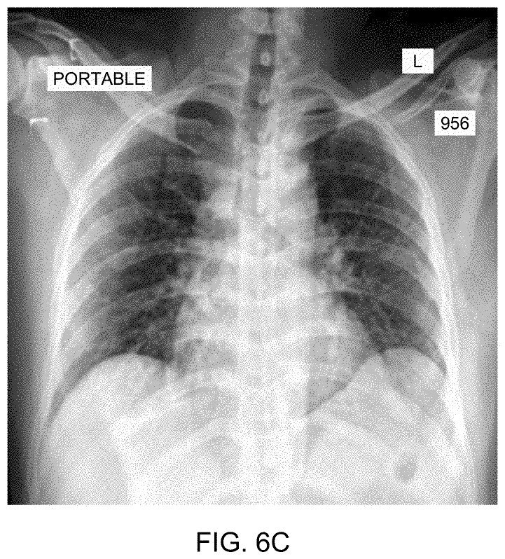

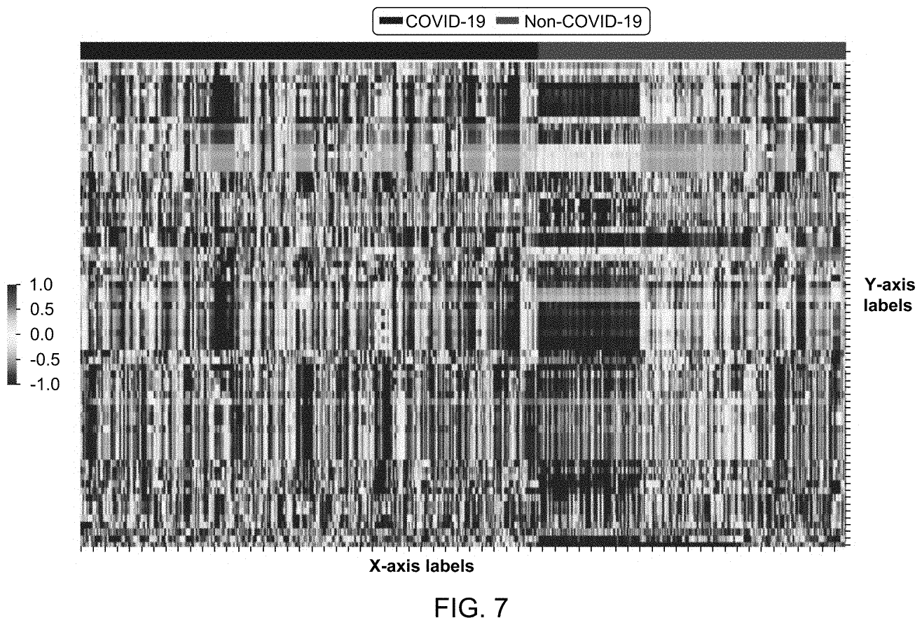

A reason for considering severity is that it is difficult to detect abnormality at low severity as shown in FIGS. 6A, 6B, 6C. FIG. 6B is a lung image for positive COVID-19, where the severity score for the right lung is 4, while the severity score for the left lung is 3. Normal lung, FIG. 6A, and a lung with very low severity score, FIG. 6C (scored 0 severity by the radiologists) appear similar though the RT-PCR test confirmed COVID-19 positive. Such low severity makes it difficult for human observer to detect abnormality in the lung in CXR.

Test data set based on severity score is shown in Table 3. It is noted that severity was not taken in to consideration during training. In S420, the machine learning model performs classification for each severity level.

TABLE 3
Number of segmented lungs for each seventy

| | Severity | | | | | |
|---|---|---|---|---|---|---|
| | 0 | 1 | 2 | 3 | 4 | Total |
| non-COVID-19 | 50 | 17 | 22 | 11 | 0 | 100 |
| COVID-19 | 36 | 88 | 71 | 27 | 8 | 230 |
| Total | 86 | 105 | 93 | 38 | 8 | 330 |

Validation on Test Data Set

After testing the machine learning model, similar to the training data set, 100 radiomics features were first extracted from each lung segment of each C×R image belonging to the test data set. However, only the 71 features that showed statistically significant difference between COVID-19 and other lung disease cases during training were used for performance evaluation. In other words, 71 extracted features were used for training, testing and verification. The three best performing machine learning algorithms during training were then applied using these 71 features to evaluate the classification performance of the machine learning algorithms using sensitivity, specificity and accuracy along with AUC-ROC. The performance was also evaluated on each separate lung to investigate the effects of severity.

### Examples

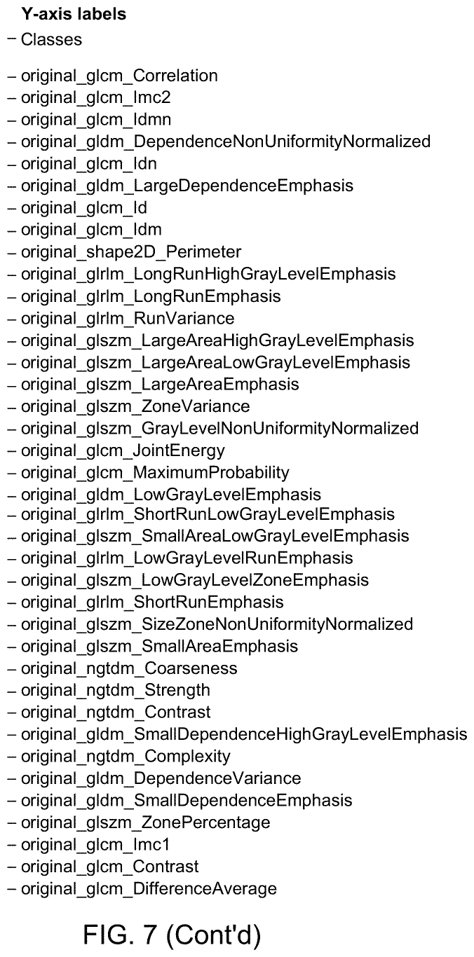

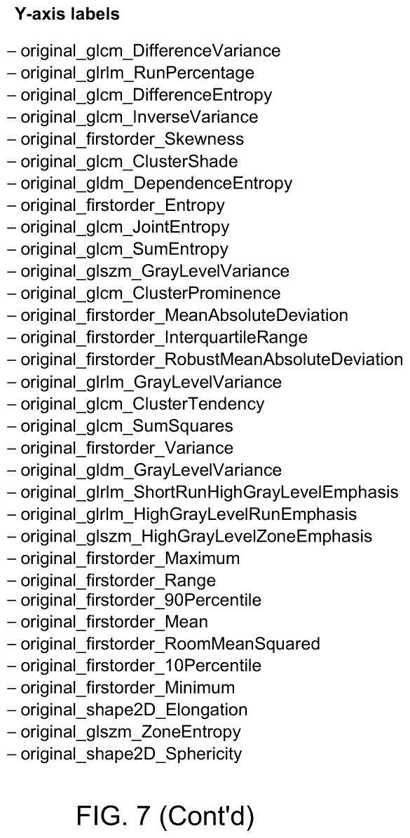

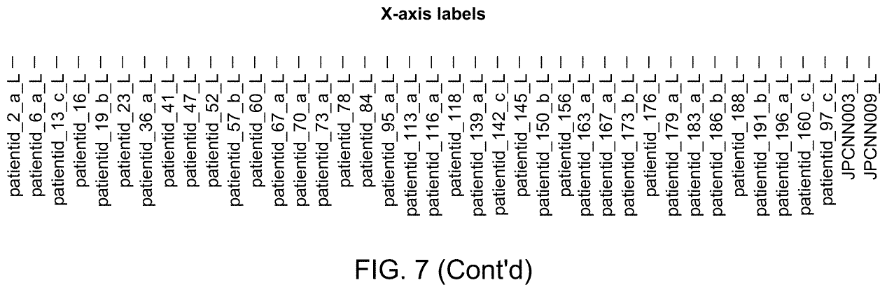

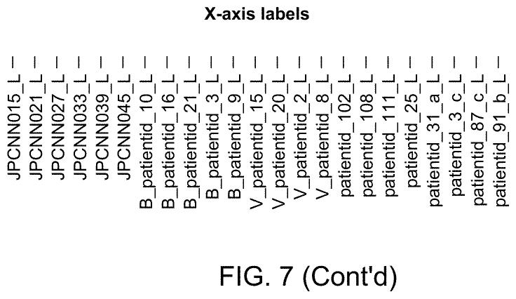

The list of the 71 radiomics features that are statistically significantly different between COVID-19 and non-COVID-19 C×R images are provided in Table 2 along with the p-value. FIG. 7 shows the Z-score heatmap of the significant radiomics features for each CXR image ordered according to the diagnosis class. The selected features clearly display the difference between the COVID-19 and other lung disease cases.

TABLE 2
p-value of the significant features extracted using ANOVA

| Feature No. | Feature Name | p |
|---|---|---|
| 1 | Firstorder 10 Percentile | 0.045053 |
| 2 | Firstorder 90 Percentile | 1.73E−05 |
| 3 | Firstorder Entropy | 0.00967319 |
| 4 | Firstorder Interquartile Range | 0.00146048 |
| 5 | Firstorder Maximum | 1.06E−10 |
| 6 | Firstorder Mean | 0.01466991 |
| 7 | Firstorder Mean Absolute Deviation | 0.00119999 |
| 8 | Firstorder Minimum | 0.02584477 |
| 9 | Firstorder Range | 5.79E−08 |
| 10 | Firstorder Robust Mean Absolute Deviation | 0.00123508 |
| 11 | Firstorder Root Mean Squared | 0.00452603 |
| 12 | Firstorder Skewness | 1.06E−10 |
| 13 | Firstorder Variance | 0.00027519 |
| 14 | GLCM Cluster Prominence | 0.00019394 |
| 15 | GLCM Cluster Shade | 1.23E−09 |
| 16 | GLCM Cluster Tendency | 0.00029227 |
| 17 | GLCM Contrast | 5.41E−10 |
| 18 | GLCM Correlation | 1.24E−05 |
| 19 | GLCM Difference Average | 1.55E−10 |
| 20 | GLCM Difference Entropy | 1.07E−10 |
| 21 | GLCM Difference Variance | 1.11E−10 |
| 22 | GLCM Id | 1.36E−10 |
| 23 | GLCM Idm | 1.43E−10 |
| 24 | GLCM Idmn | 5.58E−07 |
| 25 | GLCM Idn | 4.70E−10 |
| 26 | GLCM Imc1 | 1.73E−10 |
| 27 | GLCM Imc2 | 0.00092835 |
| 28 | GLCM Inverse Variance | 1.24E−10 |
| 29 | GLCM Joint Energy | 0.00063385 |
| 30 | GLCM Joint Entropy | 3.64E−07 |
| 31 | GLCM Maximum Probability | 0.00496733 |
| 32 | GLCM Sum Entropy | 8.69E−06 |
| 33 | GLCM Sum Squares | 0.00018272 |
| 34 | GLDM Dependence Entropy | 2.14E−08 |
| 35 | GLDM Dependence Non Uniformity Normalized | 2.95E−10 |
| 36 | GLDM Dependence Variance | 0.00193179 |
| 37 | GLDM Gray Level Variance | 0.0002227 |
| 38 | GLDM Large Dependence Emphasis | 1.16E−09 |
| 39 | GLDM Low Gray Level Emphasis | 0.02110404 |
| 40 | GLDM Small Dependence Emphasis | 0.00349533 |
| 41 | GLDM Small Dependence High Gray Level Emphasis | 0.0017444 |
| 42 | GLRLM Gray Level Variance | 0.00084841 |
| 43 | GLRLM High Gray Level Run Emphasis | 0.00143554 |
| 44 | GLRLM Long Run Emphasis | 3.97E−06 |
| 45 | GLRLM Long Run High Gray Level Emphasis | 8.93E−06 |
| 46 | GLRLM Low Gray Level Run Emphasis | 0.00189904 |
| 47 | GLRLM Run Percentage | 6.57E−09 |
| 48 | GLRLM Run Variance | 1.84E−06 |
| 49 | GLRLM Short Run Emphasis | 5.90E−06 |
| 50 | GLRLM Short Run High Gray Level Emphasis | 0.00071359 |
| 51 | GLRLM Short Run Low Gray Level Emphasis | 1.06E−10 |
| 52 | GLSZM Gray Level Non Uniformity Normalized | 1.25E−10 |
| 53 | GLSZM Gray Level Variance | 6.25E−09 |
| 54 | GLSZM High Gray Level Zone Emphasis | 0.00035915 |
| 55 | GLSZM Large Area Emphasis | 0.00239442 |
| 56 | GLSZM Large Area High Gray Level Emphasis | 0.01690471 |
| 57 | GLSZM Large Area Low Gray Level Emphasis | 0.00048366 |
| 58 | GLSZM Low Gray Level Zone Emphasis | 1.23E−05 |
| 59 | GLSZM Size Zone Non Uniformity Normalized | 1.06E−10 |
| 60 | GLSZM Small Area Emphasis | 1.06E−10 |
| 61 | GLSZM SmallAreaLowGrayLevelEmphasis | 1.06E−10 |
| 62 | GLSZM Zone Entropy | 1.06E−10 |
| 63 | GLSZM Zone Percentage | 0.02236129 |
| 64 | GLSZM Zone Variance | 0.00243722 |
| 65 | NGTDM Coarseness | 4.01E−06 |
| 66 | NGTDM Complexity | 3.41E−08 |
| 67 | NGTDM Contrast | 9.77E−09 |
| 68 | NGTDM Strength | 1.45E−10 |
| 69 | Shape2D Elongation | 1.23E−07 |
| 70 | Shape2D Perimeter | 1.30E−10 |
| 71 | Shape2D Sphericity | 1.06E−10 |

The performance of the machine learning algorithms during training is shown in Table 4. Each performance value shown in the table is the average of the 10-fold cross-validation results. From the table, it can be seen that the SVM classifier algorithm has the highest average sensitivity and AUC-ROC. But the lowest specificity. The highest specificity is achieved by fine KNN with 97.9% but with the lowest average sensitivity 88.9% and AUC-ROC of 0.9343. The sensitivity, specificity and AUC-ROC for EBM method is always more than 90%.

TABLE 4
Performance of the classifiers during training.

| Classifier | Sensitivity | Specificity | Accuracy | AUC-ROC |
|---|---|---|---|---|
| Fine Gaussian SVM | 98.2% | 88.4% | 93.4% | 0.9894 |
| Fine KNN | 88.9% | 97.9% | 93.3% | 0.9343 |
| Ensemble Bagged | 91.6% | 92.6% | 91.8% | 0.9772 |
| Model Trees (EBM) | | | | |

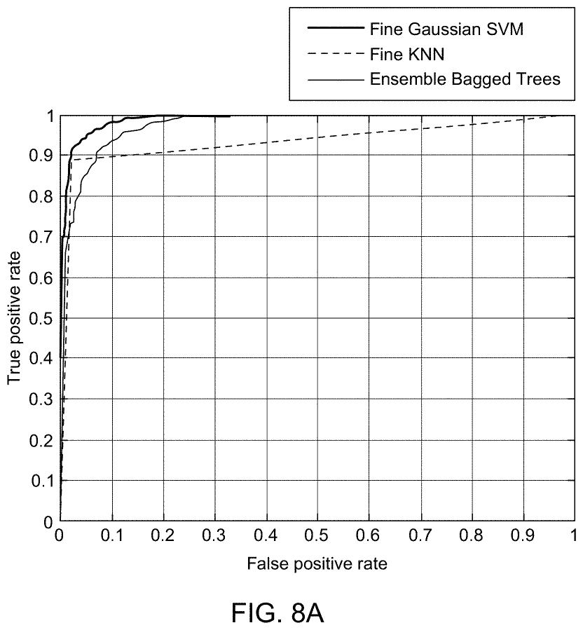

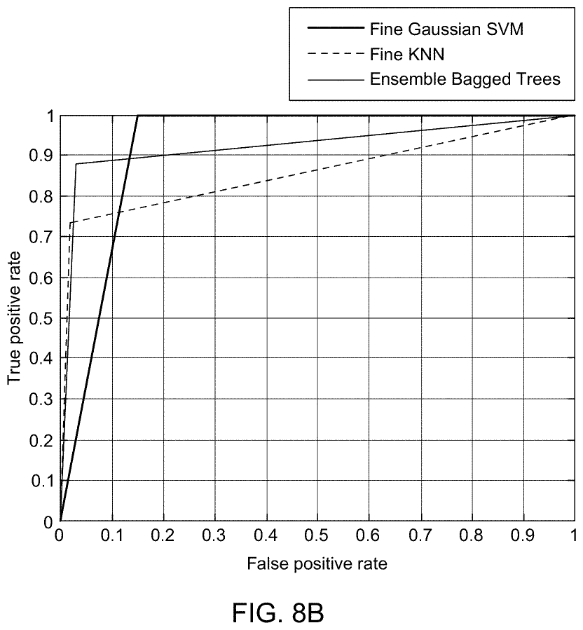

Table 5 shows the performance results of applying the previously trained machine learning algorithms to an independent test data set. FIGS. 8A and 8B show the AUC of all three machine learning algorithms during training and testing phases.

TABLE 5
Performance of the classifiers during testing.

| Classifier | Sensitivity | Specificity | Accuracy | AUC-ROC |
|---|---|---|---|---|
| Fine Gaussian SVM | 99.6% | 85% | 95.2% | 0.9228 |
| Fine KNN | 73.5% | 98% | 80.9% | 0.8574 |
| Ensemble Bagged | 87.8% | 97% | 90.6% | 0.9241 |
| Model Trees (EBM) | | | | |

The SVM classifier algorithm was able to correctly predict all the COVID-19 cases except one with 99.6% sensitivity, while EBM correctly predicted COVID-19 with 87.8% sensitivity. The sensitivity of the fine KNN was the lowest with 73.5%. On the other hand, the two highest specificity of 98% and 97% were achieved by KNN and EBM respectively, while the specificity of the SVM method was the lowest with 85%. The highest accuracy and AUC-ROC were achieved by SVM and EBM respectively (95.2% and 0.9241).

The ROC curves of all the classifiers during training and test cases are shown in FIG. 8A, 8B.

Table 6 and 7 compare sensitivity and specificity of SVM and EBM classifiers respectively based on the severity. Since the accuracy and AUC-ROC of the fine KNN were less than 90% and 0.9 respectively, it was not considered for further investigation. This is to be noted that for some COVID-19 patients, some of the lung segments were scored as 0 by the radiologists. On the other hand, there were no lung segments with severity score 4 for viral/bacterial pneumonia.

TABLE 6
Sensitivity and specificity based on severity for SVM

| | | Severity | | | | | |
|---|---|---|---|---|---|---|---|
| | | 0 | 1 | 2 | 3 | 4 | Total |
| COVID-19 | Original | 36 | 88 | 71 | 27 | 8 | 230 |
| | Detected | 35 | 88 | 71 | 27 | 8 | 229 |
| | Sensitivity | 97.2% | 100.0% | 100.0% | 100.0% | 100.0% | 99.6% |
| Non-COVID-19 | Original | 50 | 17 | 22 | 11 | 0 | 100 |
| | Detected | 47 | 11 | 20 | 7 | 0 | 85 |
| | Specificity | 94.0% | 64.7% | 90.9% | 63.6% | 0.0% | 85.0% |

TABLE 7
Sensitivity and specificity based on severity for EBM.

| | | Severity | | | | | |
|---|---|---|---|---|---|---|---|
| | | 0 | 1 | 2 | 3 | 4 | 5 |
| COVID-19 | Original | 36 | 88 | 71 | 27 | 8 | 230 |
| | Detected | 33 | 78 | 64 | 21 | 6 | 202 |
| | Sensitivity | 91.7% | 88.6% | 90.1% | 77.8% | 75.0% | 87.8% |
| Non-COVID-19 | Original | 50 | 17 | 22 | 11 | 0 | 100 |
| | Detected | 50 | 16 | 20 | 11 | 0 | 97 |
| | Specificity | 100.0% | 94.1% | 90.9% | 100.0% | 0.0% | 97.0% |

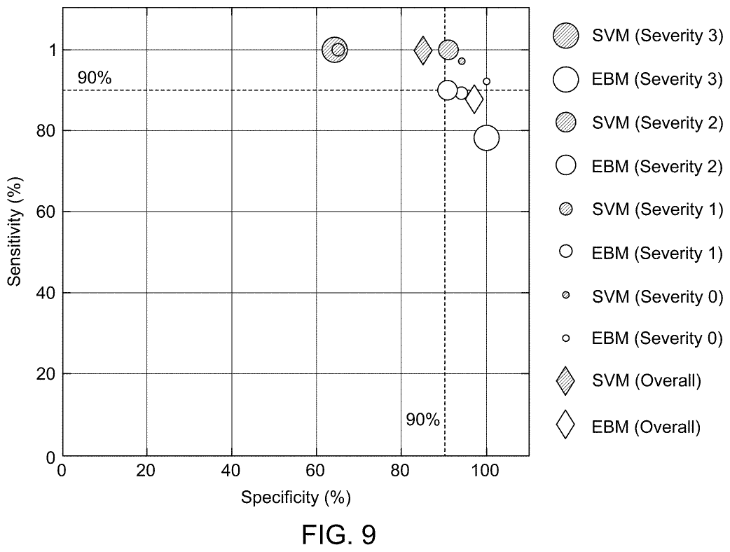

Sensitivity vs. specificity for both SVM and EBM for the whole test data and at each severity level is plotted in FIG. 9.

Overall, SVM can detect COVID-19 with 99.6% sensitivity and 85% specificity. On the other hand, the performance of EBM is 87.8% and 97% respectively. For the EBM method, the sensitivity decreases with the increase in severity. On the other hand, the sensitivity of the SVM methods is less dependent on the severity level. However, high level of fluctuations is observed in terms of specificity.

This implies that there are certain radiomics features that are different for COVID-19 patients and these features are dependent on severity level. Appropriate selection of those features also can allow detection of COVID-19 from other lung diseases.

For the whole test data set, the performance of SVM and EBM was comparable. However, KNN performance was much worse compared to the training phase. The reason behind it could be that during training only average sensitivity and specificity of the 10 fold validation were calculated and due to data augmentation via geometric transformation.

The performance difference between SVM and EBM methods are more distinguishable if severity is taken into consideration. SVM method shows very good sensitivity (97.2 to 100%) but the specificity in terms of severity is not robust with values ranging from 63.6 to 94%. On the other hand, the sensitivity of EBM decreases with the increase of severity. The specificity of the EBM method is within 10% range for all levels of severity and never falls below 90% (range 90.9 to 100%).

The disclosed method is a rapid diagnosis tool to detect COVID-19 from CXR. Once the CXR is acquired and lung is segmented, it takes less than two minutes for radiomics features extraction on an Intel Core i7 1.5 GHz 4 cores machine with 16 GB RAM and Windows 10 (64-bit) operating system. For classification SVM, KNN and EBM take 1.46, 0.86 and 1.94 seconds respectively on the same machine. Overall time required is less than 2 minutes and no preprocessing steps are involved.

Several studies have been proposed to use different machine learning algorithms to detect COVID-19 from C×R images. See Ahmed et al., 2020; Asif et al., 2020; and Ozturk, T., Talo, M., Yildirim, E. A., Baloglu, U. B., Yildirim, O., & Rajendra Acharya, U. (2020). Automated detection of COVID-19 cases using deep neural networks with X-ray images. Computers in Biology and Medicine, 121, Article 103792, each incorporated herein by reference in their entirety. Most of them have demonstrated good accuracy for disease diagnosis. The performance of those methods vary depending on the definition of the classes used to determine the accuracy. The highest accuracy of 98.08% was achieved by deep learning method (Ozturk et al., 2020). However, it only uses two data classes—COVID-19 and No-Findings. Inclusion of multiple diseases brings the accuracy down to 87.02%. Other studies also reported to achieve similar sensitivity (93 to 96.7%) and specificity (90 to 100%) to classify between COVID-19 and others class, where other class consists of only normal CXR and viral pneumonia. To increase the number of images (sometimes as many as 10 times of the original data), all these algorithm mainly used geometric transformation for data augmentation. Training and test data set were then randomly split. As a result, it is highly likely that the images of the same patients are present in both the training and test sets resulting in higher accuracies in detection of COVID-19.

A robust method should be able to detect COVID-19 in presence or absence of other possible lung conditions and the performance should not fluctuate considerably for any other independent data set. From this perspective, the disclosed method is robust as it not only includes normal and viral pneumonia but also other lung diseases (e.g., bacterial pneumonia, SARS etc.) in the training data as shown in Table 1. The performance is also tested on a completely independent data set. The other uniqueness of the disclosed method is that it can provide similar performance irrespective of the severity levels. Performance evaluation based on severity reveals more insight into the radiomics pattern present in CXR images. Visual assessment of 36 lungs by the radiologists reveals no abnormality and were assigned with the severity score of 0. However, RT-PCR test confirmed COVID-19 positive for these lungs and the disclosed method also confirmed COVID-19 positive with a sensitivity of 97.2% and 91.7% by SVM and EBM method respectively. Thus, the disclosed method can detect COVID-19 from CXR even when experienced radiologists are unable to detect any abnormality in the lung CXR (represented by severity score of 0).

Considering the performance at different severity levels, EBM method proves to be the most robust method. However, the sensitivity of the EBM method decreases with the increase of severity. One of the reasons could be that with the increase of severity, the patterns that serve as radiomics features of COVID-19 vanish and become less distinguishable compared to other lung conditions.

Unlike other studies dealing with the diagnosis of COVID-19 from CXR, the performance of the disclosed method was validated on a completely independent data set and considering different levels of disease severity. Disclosed embodiments may also be applied to cases in which RT-PCR is negative at an early time point but becames positive later.

With appropriate selection of radiomics features and machine learning algorithm, it is possible to detect COVID-19 directly from CXR with a sensitivity and specificity comparable or better than other available techniques, e.g., RT-PCR. The performance of the disclosed method with SVM and EBM based machine learning achieved an overall sensitivity of 99.6% and 87.8% and specificity of 85% and 97% respectively. Though the performance are comparable for both the methods, EBM is more robust across severity levels. Since this tool does not require any manual intervention (e.g., sample collection etc.), it can be integrated with any standard X-ray reporting system as an efficient, cost-effective and rapid point-of-care device.

1. A method for diagnosis of COVID-19 from at least one Chest X-Ray image, comprising:
imaging, by a chest x-ray machine, a person's chest area to obtain the at least one chest x-ray image;
performing, by processing circuitry, image segmentation of a human lung in the at least one chest x-ray image;
extracting, by the processing circuitry, radiomics features from the segmented lung;
selecting, by the processing circuitry, a subset of the radiomics features for classification ability between two classes of COVID-19 and non-COVID-19 including other lung diseases, wherein the subset of radiomics features includes: 13 first order, 3 2D shape based, 20 Gray Level Co-occurrence Matrix (GLCM), 8 Gray Level Departure Matrix (GLDM), 10 Gray Level Run Length Matrix (GLRLM), 13 Gray Level Size Zone Matrix (GLSZM) and 4 Neighboring Gray Tone Difference Matrix (NGTDM) features;
classifying, by the processing circuitry, between COVID-19 and non-COVID-19 using an ensemble bagged model having a plurality of classifiers; and
outputting, by the processing circuitry, an indication of whether the patient is infected with COVID-19.

2. The method for diagnosis of COVID-19 of claim 1, wherein the selecting of radiomics features includes producing a heatmap of Z-scores for the radiomics features to identify the subset of radiomics features that significantly classify the two classes of COVID-19 and other lung diseases.

3. The method for diagnosis of COVID-19 of claim 1, wherein the selecting of radiomics features includes determining a one-way analysis of variance test to find the subset of features that have a statistically significant difference between means of the two classes, with criteria p<0.05, where p-value is a probability.

4. The method for diagnosis of COVID-19 of claim 1, wherein the plurality of classifiers for the ensemble bagged model are decision trees.

5. The method for diagnosis of COVID-19 of claim 1, wherein the imaging, by a chest x-ray machine, is performed for a plurality of different persons to obtain a plurality of chest x-ray images for the different persons, and
wherein the plurality of chest x-ray images are grouped by a level of severity based on the extent of involvement by ground glass opacities, and the classifying is separately performed for each group.

6. A mobile device for diagnosis of COVID-19 from at least one Chest X-Ray image, comprising:
a display device; and
processing circuitry configured to:
perform image segmentation of a human lung in the at least one Chest X-Ray image;
extract radiomics features from the segmented lung;
select a subset of the radiomics features for classification ability between two classes of COVID-19 and non-COVID-19 including other lung diseases, wherein the subset of the radiomics features includes: 13 first order, 3 2D shape based, 20 Gray Level Co-occurrence Matrix (GLCM), 8 Gray Level Departure Matrix (GLDM), 10 Gray Level Run Length Matrix (GLRLM), 13 Gray Level Size Zone Matrix (GLSZM) and 4 Neighboring Gray Tone Difference Matrix (NGTDM) features;
classify between COVID-19 and non-COVID-19 using an ensemble bagged model having a plurality of classifiers; and
output to the display device an indication of whether the patient is infected with COVID-19.

7. The mobile device of claim 6, wherein the processing circuitry is further configured to produce a heatmap of Z-scores for the radiomics features to identify the subset of radiomics features that significantly classify the two classes of COVID-19 and other lung diseases.

8. The mobile device of claim 6, wherein the processing circuitry is further configured to determine a one-way analysis of variance test to find the subset of features that have a statistically significant difference between means of the two classes, with criteria p<0.05, where p-value is a probability.

9. The mobile device of claim 6, wherein the plurality of classifiers for the ensemble bagged model are decision trees.

10. The mobile device of claim 6, further comprising:
communication circuitry configured to wirelessly communicate with at least one chest x-ray machine to receive the at least one chest x-ray image.

11. The mobile device of claim 10, wherein the communication circuitry is configured to wirelessly communicate with a plurality of chest x-ray machines to receive a respective plurality of chest x-ray images for a plurality of patients,
wherein the processing circuitry is further configured to
perform image segmentation of a human lung in each of the plurality of chest X-ray images;
extract radiomics features from the segmented lungs;
select a subset of the radiomics features for classification ability between two classes of COVID-19 and non-COVID-19 including other lung diseases;
classify between COVID-19 and non-COVID-19, for each of the plurality of chest x-ray images using the ensemble bagged model having the plurality of classifiers; and
output to the display device, while simultaneously storing in a database, an indication for each of the plurality of patients whether the respective patient is infected with COVID-19.

12. A non-transitory computer readable storage medium storing program instructions, which when executed by processing circuitry performs a method for diagnosis of COVID-19 from at least one Chest X-Ray image comprising:
performing image segmentation of a human lung in the at least one Chest X-Ray image;
extracting radiomics features from the segmented lung;
selecting a subset of the radiomics features for classification ability between two classes of COVID-19 and non-COVID-19 including other lung diseases, wherein the subset of radiomics features includes: 13 first order, 3 2D shape based, 20 Gray Level Co-occurrence Matrix (GLCM), 8 Gray Level Departure Matrix (GLDM), 10 Gray Level Run Length Matrix (GLRLM), 13 Gray Level Size Zone Matrix (GLSZM) and 4 Neighboring Gray Tone Difference Matrix (NGTDM) features;
classifying between COVID-19 and non-COVID-19 using an ensemble bagged model having a plurality of classifiers; and
outputting an indication of whether the patient is infected with COVID-19.

13. The non-transitory computer readable storage medium of claim 12, wherein the method further includes producing a heatmap of Z-scores for the radiomics features to identify the subset of radiomics features that significantly classify the two classes of COVID-19 and other lung diseases.

14. The non-transitory computer readable storage medium of claim 12, wherein the method further includes determining a one-way analysis of variance test to find the subset of features that have a statistically significant difference between means of the two classes, with criteria p<0.05, where p-value is a probability.
